(19) United States
(12) Patent Application Publication
(10) Pub. No.: US 2022/0170069 A1
(43) Pub. Date: Jun. 2, 2022

(54) DEVICE FOR DETERMINING THE PRESENCE OF A BACTERIOLOGICAL CONTAMINATION IN A FLUID

(71) Applicant: BIOMÉRIEUX, Marcy L'Etoile (FR)

(72) Inventors: Yoann BUATHIER, Caluire et Cuire (FR); Emilie HEURTAUX, Annecy (FR); Marie-Pierre MONTET, Venissieux (FR); Christine ROZAND, Saint Genis Les Ollieres (FR); Cécile THEVENOT, Labarthe sur Leze (FR)

(73) Assignee: BIOMÉRIEUX, Marcy L'Etoile (FR)

(21) Appl. No.: 17/601,158

(22) PCT Filed: Mar. 31, 2020
(86) PCT No.: PCT/FR2020/000076

§ 371 Date: Oct. 4, 2021

(30) Foreign Application Priority Data
Apr. 9, 2019 (FR) 1903807
Oct. 21, 2019 (FR) 1911739

(51) Int. Cl. C12Q 1/22; B01D 63/08; B01L 3/00; G01N 1/40

(52) U.S. Cl. CPC C12Q 1/22; B01D 63/087; B01L 3/5023; G01N 1/4077; G01N 21/6428; B01L 2300/0803; B01L 2300/042; G01N 2001/4088; B01D 2313/20

(57) ABSTRACT

A device for determining contamination of a fluid by microorganisms has a housing with an internal volume, a cover closing the housing, a fluid inlet port, at least one filtration member, at least one nutrient layer including a composition of a microbiological culture medium, characterized in that the device includes a fluid outlet port, and in that the cover has an inner surface in the internal volume extending radially about the fluid inlet port up to a peripheral edge of the cover, said inner surface being inclined and converging towards the fluid inlet port, and in that the bottom of the housing has a surface extending radially about the fluid outlet port up to the side wall of the housing, said inner surface being inclined and converging towards the fluid outlet port.

### TECHNICAL FIELD OF THE INVENTION

The invention pertains to the technical domain of microbiological analysis. More specifically, the invention relates to a microbiological testing device for testing a liquid to be analyzed, this liquid being liable to contain at least one microorganism.

### CONTEXT OF THE INVENTION

The invention can be applied to the domain of industrial, food, pharmaceutical, cosmetic or veterinary microbiological testing, for terrestrial or space use.

The invention has been developed following work supported by the French National Center for Space Studies (CNES) and the company INITIAL.

### TECHNOLOGICAL BACKGROUND TO THE INVENTION

There are numerous circumstances in which liquids are tested to determine the presence of at least one microorganism, usually to determine the absence of said microorganism. Naturally, the liquid to be analyzed can be a biological fluid (whole blood, serum, plasma, urine, cerebrospinal fluid, organic secretion, etc.). However, the liquid can also be an industrial liquid, notably a food liquid (water, other beverage, in particular fruit juice, milk, soft drink, etc.) or a pharmaceutical or cosmetic or veterinary liquid (milk from a sick animal).

Numerous laboratory techniques are known for filtering the liquid to be analyzed to collect any microorganisms contained in the liquid, to culture these microorganisms for subsequent detection, counting, characterization and/or identification thereof. These techniques require a certain number of actions and specific infrastructure (filtration ramp, lab benches, incubators) well known to laboratory assistants.

These techniques often require the use of a filtration device that includes a closed internal space delimited by an enclosure that is designed to receive the liquid to be analyzed. Such a technique is notably referred to as “membrane filtration”. Microbiological filtering means, for example a filtering membrane, are arranged in the closed internal space and separate, in the closed internal space, a first compartment from a second compartment of the closed internal space. The device (filter cassette) has an inlet port for the liquid to be analyzed that opens into the first compartment of the closed internal space.

In some known filtration devices, the device is opened to retrieve the filtering means, which are then transferred to a culture device to incubate the microorganisms. Such techniques are easy to perform in the laboratory.

Conversely, such techniques are difficult to implement in operational environments, on the ground, in the International Space Station, and in industrial environments in which liquids are produced, packaged, distributed or used.

A microbiological testing device and a method for testing a liquid to be analyzed that enable particularly simplified testing operations that can be used or performed away from a microbiological laboratory, including in an industrial environment, are known from document WO2018189478 A1. This document describes a microbiological testing device for testing a liquid to be analyzed that is liable to contain at least one microorganism. The testing device comprises a closed housing designed to receive the liquid to be analyzed, microbiological filtering means arranged in the closed internal space that separate, in the closed internal space, a first compartment from a second compartment of the closed internal space, an inlet port for the liquid to be analyzed that opens into the first compartment of the closed internal space, a nutrient layer comprising a microbiological culture medium. The main drawback of this device is the fact that the liquid remains in the device following filtration and cannot be recycled, and that the liquid remaining in the device can leach the PAD, dilute the nutrient layer and generate false negatives. Furthermore, manufacture of the device requires the housing to be placed in a vacuum, which is a significant constraint.

### Purpose of the Invention

The invention is an improvement of the aforementioned device, that is more compact and more practical.

For this purpose, the invention relates to a device for determining microorganism contamination of a fluid, said device comprising:
a housing having an internal volume delimited by at least one side wall and a bottom,
a cover closing the housing and positioned opposite the bottom,
a fluid inlet port arranged on the cover that opens out into the internal volume of said housing, the fluid inlet port extending along an axis that is substantially secant and preferably perpendicular to the cover,
at least one filtration member arranged in the internal volume,
at least one nutrient layer comprising a microbiological culture medium,
   characterized in that the device includes:
a fluid outlet port arranged on the housing that opens out into the internal volume of said housing, and in that the cover has an inner surface in the internal volume extending radially about the fluid inlet port up to the peripheral edge of the cover (3), said inner surface being inclined or curved and converging towards the fluid inlet port,
a support grille configured to support the nutrient layer
and in that the bottom of the housing has a surface extending radially about the fluid outlet port up to the side wall of the housing, said surface being inclined and converging towards the fluid outlet port.

Advantageously, in the device according to the invention, the fluid passes through the device and does not stagnate in the bottom of the housing and on the filter, which ensures that the liquid passes quickly through the device, requiring less force to do so, prevents leaching of the nutrient layer, and ensures that the microorganisms are collected under optimum conditions and incubated under optimum conditions. Furthermore, the inclination of the inner surface of the cover helps to improve distribution of the fluid in the device and ensures that all of the fluid is distributed uniformly over the filter to ensure a uniform distribution of microorganisms on the filter, thereby preventing the formation of any microbial agglomerations on the filter, which could generate false negatives.

Advantageously, the device is designed to quantify and detect two fecal contaminants, for example E. coli and enterococcus bacteria.

In the present invention, the term “port” means a fluid channel with a central orifice passing longitudinally through said channel.

The device according to the invention is preferably used vertically, i.e. the fluid inlet port and the fluid outlet port are positioned vertically.

In the present application, an object positioned vertically is an object arranged in a direction parallel to the direction of gravity.

According to one feature of the invention, the fluid is a liquid, for example water, or the fluid is a gas, for example air.

According to another feature of the invention, the inclination of the inner surface of the cover is strictly greater than +4°. Preferably, the inclination of the inner surface of the cover is preferably between +5° and +15°, and more preferably substantially 10°.

According to another feature of the invention, the inclination of the surface of the bottom is strictly less than 0°. Preferably, the inclination of the surface of the bottom is preferably between −5° and −10°, and more preferably between −6.5° and −7.5°.

According to another feature of the invention, the fluid inlet port projects into the internal volume of the housing relative to the inner surface of the cover.

Preferably, the fluid inlet port is arranged substantially in the center of the cover.

According to another feature of the invention, the inlet port has a first end extending outside the cover that is designed to cooperate with a stopper.

According to another feature of the invention, the fluid inlet port has a second end extending inside the internal volume, the second end having at least one lateral orifice opening out into the internal volume.

According to another feature of the invention, the at least one lateral orifice is oriented to at least partially spray the fluid towards the inner surface of the cover.

According to another feature of the invention, the inlet port has at least two lateral orifices, preferably arranged opposite one another.

According to another feature of the invention, the fluid outlet port has a first end extending outside the housing that is designed to cooperate with a stopper.

According to another feature of the invention, the fluid inlet port has a second end that is flush with the bottom of the housing.

According to a feature of the invention, the filtration member is arranged in the housing at a given distance h from the inlet port. Advantageously, the given distance h is strictly greater than 1 mm, and preferably between 1.5 mm and 5 mm, and more preferably between 2.5 mm and 3.5 mm. The filtration member must be at a given distance h so that the microorganisms can grow and form easily visible colonies when the device is incubated. This given distance is dependent on a minimum volume of air required to grow said microorganisms.

According to another feature of the invention, when the inlet port and the fluid outlet port are blocked by the stoppers, the device according to the invention is fluid tight.

According to a feature of the invention, the cover is transparent or translucent, which enables the microbial colonies to be viewed after incubation.

According to a feature of the invention, the housing is substantially cylindrical or polygonal.

According to a feature of the invention, the housing has a first circumferential bearing surface shaped to cooperate complementarily with an edge of the cover. Advantageously, the cover and the housing are welded together by ultrasound.

According to a feature of the invention, the housing has at least one second bearing surface extending in the internal volume and shaped to receive a peripheral portion of the filtration member, said second bearing surface being shaped to cooperate with a portion of the cover such that the peripheral portion of the filtration member is clamped between the cover and the housing preferably uniformly so that the filtration member is stable and static as the fluid passes through the device, to ensure there are no leaks and that all of the fluid passes through the filtration member.

According to another feature of the invention, the bottom of the housing has supporting ribs arranged radially relative to the fluid outlet port.

According to a feature of the invention, said ribs are designed to hold the support grille and to guide the fluid towards the fluid outlet port.

According to a feature of the invention, the supporting ribs project from the surface of the bottom and extend substantially perpendicularly to said surface of the bottom.

According to a feature of the invention, the internal pressure of the housing is substantially equal to the pressure outside the housing under terrestrial conditions, i.e. when no depression is applied before sealing the cover on the housing.

According to a feature of the invention, the support grille is designed to support the nutrient layer.

According to a feature of the invention, the support grille is also designed to support the filtration member.

According to a feature of the invention, the support grille is arranged between the nutrient layer and the bottom of the housing.

According to a feature of the invention, the support grille is overall cylindrical or polygonal.

According to a feature of the invention, the support grille has a plurality of through holes distributed over the entire grille, which helps to distribute the fluid that has passed through the nutrient layer over the entire surface of the bottom, in order to drain said liquid more quickly and to prevent the liquid from stagnating in the device.

According to a feature of the invention, the support grille preferably rests on the supporting ribs arranged on the bottom of the housing.

According to one feature of the invention, the support grille is preferably the same size and shape as the nutrient layer positioned above, such as to prevent any deformation of the nutrient layer during passage of the liquid.

According to one feature of the invention, the perimeter of the support grille is shaped to fit the shape of the housing so that the grille is held in the housing and in the internal volume by friction.

According to one feature of the invention, the housing has a third bearing surface 25 that is designed to receive the support grille and more specifically to receive a peripheral edge of the support grille.

According to one feature of the invention, the third bearing surface extends inside the internal volume of the housing and is an external shoulder of the housing.

According to one feature of the invention, the filtration membrane is permeable to fluids and in particular to a gas or to a liquid having a viscosity that enables microbiological filtration free of all solid particles.

According to another feature of the invention, the filtration member is a filtration member impermeable to microorganisms and preferably to bacteria, which enables same to be retained on the surface of said at least one filtration member.

According to a feature of the invention, the filtration member is distinct from the nutrient layer.

According to a feature of the invention, the filtration membrane is permeable to nutrients and to any additives included in the nutrient layer.

According to a feature of the invention, the filtration member is a porous membrane and can for example be made from one or more materials or derivatives of these materials, such as latex, polytetrafluoroethylene, poly(vinylidene) fluoride, polycarbonate, polystyrene, polyamide, polysulfone, polyethersulfone, cellulose or a mixture of celluloses and nitrocellulose.

Advantageously, the filtration member has a surface above the surface of the nutrient layer such that the surface of said nutrient layer is entirely covered by the filtration member.

Preferably, the filtration member is white or similarly colored, which helps to optimize differentiation of colored colonies on the surface thereof. Alternatively, the filtration member can be dark to facilitate visibility of white or cream-colored colonies.

Advantageously, the filtration capacity and the hydrophilia of the filtration member are used to enable and optimize passage of nutrients and of any additives in the nutrient layer following re-hydration thereof to an upper surface of the filtration member, while preventing or limiting migration of filtered bacteria, yeast and like to the upper surface of the filtration member in the opposite direction.

Advantageously, the filtration member has pores with a diameter of between 0.01 μm and 0.8 μm, preferably between 0.2 μm and 0.6 μm, such as to retain the bacteria, yeast and mold on the surface thereof. According to a specific embodiment, the filtering means have pores with a diameter of between 0.25 μm and 0.6 μm, for example between 0.3 μm and 0.6 μm, or between 0.4 μm and 0.6 μm. Alternatively, a layer with no measurable pores, such as a dialysis membrane, may be used. For example, the filtration member may be a membrane in the “Fisherbrand™ General Filtration Membrane Filters” range sold by Fisher Scientific Company L.L.C, 300 Industry Drive, Pittsburgh, Pa. 15275, USA, or a membrane in the “Nitocellulose Membrane Filters” range manufactured by Zefon International, Inc., 5350 SW 1st Lane, Ocala, Fla. 34474, USA.

According to a feature of the invention, the nutrient layer is arranged beneath the filtration member and is preferably in contact with said filtration member.

According to a feature of the invention, the nutrient layer includes a support containing a microbiological culture medium.

According to a feature of the invention, the support may be made of various absorbent compounds, preferably with a very high water-retention capacity, such as rayon, cotton, natural or chemically modified cellulose fibers such as carboxymethyl cellulose, absorbent or super-absorbent chemical polymers such as polyacrylate salts, acrylate/acrylamide copolymer.

According to another feature of the invention, the support can be impregnated with a microbiological culture medium in liquid form.

Advantageously, the microbiological culture medium can advantageously be dehydrated, i.e. having an activity of water (Aw) that is incompatible with microbial development. Alternatively, the support can be covered or impregnated dry with a microbiological culture medium or the ingredients thereof in powder form. Alternatively, liquid impregnation can be carried out by adding powder after dehydration.

Microbiological culture medium means a medium including the nutrients required for the survival and/or growth of microorganisms, notably one or more of the following: carbohydrates, including sugars, peptones, growth promotors, mineral salts and/or vitamins, etc. In practice, the person skilled in the art will choose the microbiological culture medium as a function of the target microorganisms, depending on well-known criteria available to said person skilled in the art. The nutrient layer can contain additives such as:
one or more selective agents such as inhibitors or antibiotics to encourage the growth and development of one species/strain of a specific organism rather than another,
buffers, colorants.

In general, the nutrient layer can also contain a substrate used to detect enzymatic or metabolic activity in the target microorganisms using a signal detectable directly or indirectly. For direct detection, this substrate can be linked to a part acting as a fluorescent or chromogenic marker. For indirect detection, the nutrient layer according to the invention can also include a pH indicator that is sensitive to pH variations caused by consumption of the substrate, revealing growth of the target microorganisms. Said pH indicator can be a chromophore or a fluorophore. Neutral red, aniline blue and bromocresol blue are example chromophores. 4-Methylumbelliferone, derivatives of hydroxycoumarin and derivatives of resorufin are all fluorophores. Thus, the PC-PLC fluorescent substrate preferably used to implement the method according to the invention is 4-Methyl-Umbelliferyl-Choline Phosphate (4 MU-CP).

According to one feature of the invention, the microbiological culture medium of the nutrient layer is dehydrated in a delivery configuration of the device according to the invention before use. In this case, following dry impregnation of the support of the nutrient layer by the dehydrated microbiological culture medium, the nutrient layer can undergo a calendering operation. The pressure and the heat generated by the calendering enables the dehydrated microbiological culture medium in the support of the nutrient layer to be retained and kept stable over time, ensuring retention of the nutrients and any additives in the nutrient layer. The calendering of the nutrient layer also helps to ensure that the surface of the nutrient layer is flat and smooth. The calendering also helps to accelerate re-hydration of the nutrient layer relative to a nutrient layer that is not calendered, on account of the resulting compression of the nutrient layer. If the support is made of fibers, this compression, which is associated with the presence of the dehydrated medium in the nutrient layer, generates a significant increase in the capillary capacity of this latter, causing the near-instant re-hydration thereof. This can also contribute to an aspiration phenomenon of the separate microbiological filtration member arranged against the surface thereof. The microbiological filtration member can thus be pressed against the nutrient layer, thereby eliminating or reducing the space between the two, and ensuring optimal microbial growth and/or survival on the surface of the microbiological filtering means. This helps to obviate the need for linking means (for example obviating the need for a linking layer) between the microbiological filtration member and the nutrient layer when these are separate. This represents a significant advantage since such linking means would slow the passage of the nutrients and any additives from the rehydrated nutrient layer to the microorganisms present on the microbiological filtering means, thereby reducing the growth and/or survival chances of these microorganisms.

Preferably, the nutrient layer can be the microbiological culture device described in patent application FR19/03751 Filed on Apr. 8, 2019.

According to a feature of the invention, the nutrient layer is a microbiological culture device including some or all of a dehydrated microbiological culture medium in powder form, having at least two parts made of absorbent hydrophilic material with an upper face that is at least substantially flat, with said dehydrated microbiological culture medium in powder form being arranged between two contiguous parts, and said microbiological culture medium including at least one gelling agent in powder form.

Numerous absorbent, hydrophilic and non-water-soluble materials can be used to make the absorbent-material part of a microbiological culture device according to the invention. These materials are primarily selected as a function of absorption capacity, capacity to retain aqueous liquids and ability to enable aqueous liquids to pass therethrough in all directions.

According to a feature of the invention, the absorbent-material part is made from a substrate of short nonwoven fibers forming an assembly providing structural integrity and mechanical consistency. Particularly suitable substrates are made of natural cellulose fibers (such as cotton) or synthetic cellulose fibers (such as rayon), modified cellulose fibers (for example carboxymethyl cellulose and nitrocellulose), and absorbent chemical polymer fibers (such as polyacrylate salts and acrylate/acrylamide copolymers). Advantageously, the absorbent material part is made of non-woven textile made of cellulose fibers.

In the present invention, the absorbent hydrophilic material parts of a given device can have the same mass density or different mass densities. Similarly, the absorbent hydrophilic material parts of a given microbiological culture device according to the invention can have the same thickness or different thicknesses.

According to one feature of the invention, the absorbent hydrophilic material part has a mass density of between 0.045 g/cm³ and 0.10 g/cm³, and preferably between 0.05 g/cm³ and 0.07 g/cm³.

The absorbent hydrophilic material parts of a given microbiological culture device according to the invention can have a thickness of between 0.5 mm and 2 mm. Preferably, the absorbent hydrophilic material part has a thickness of 0.8 mm to 1.8 mm, and more preferably between 1 mm and 1.5 mm. The surface of the absorbent hydrophilic material part is between 1 cm² and 40 cm², preferably between 10 cm² and 30 cm², and more preferably between 15 cm² and 25 cm².

According to a feature of the invention, the absorbent hydrophilic material part is able to retain the volume of water greater than 2 mL, preferably greater than 3 mL. Thus, a porous support having a 25 cm² surface and a thickness of 1 mm after calendering will be able to hold 3 mL of water.

According to a feature of the invention, the microbiological culture device has undergone a calendering operation. The pressure and heat generated by calendering enables the dehydrated reaction medium including the gelling agent or agents in the microbiological culture device to be retained and kept stable over time, ensuring retention of the different elements such as nutrients between the absorbent hydrophilic material parts.

Preferably, calendering is performed at a temperature greater than ambient temperature, preferably at a temperature of between 30° C. and 60° C. Temperatures below 60° C. ensure that the thermolabile compounds in the culture medium are not denatured.

Calendering also ensures that the reaction medium is kept within the microbiological culture device, thereby facilitating handling thereof.

According to another feature of the invention, the culture medium also includes at least one gelling agent, also in powder form. Once activated and re-hydrated by a liquid to be analyzed, the gelling agent or agents help to create a whole with a certain degree of structural integrity and mechanical consistency. Furthermore, by coming into contact with the culture medium, the gelling agent or agents provide the microbiological culture device with a gelatinous consistency that encourages the establishment of microorganisms and enables the elements making up the culture medium, such as nutrients or active agents, to be as close as possible to the microorganisms.

According to another feature of the invention, the gelling agent is one of the following: xanthan gum, alginate, gellan gum, galactomannan gum, locust bean gum, starch, or a mixture thereof.

Preferably, the culture medium of the device according to the invention includes between 0.0030 g/cm³ and 0.020 g/cm³ of at least one gelling agent.

The invention also relates to an assembly comprising at least one fluid feed and at least one device according to the invention, said device being connected by the fluid inlet port to a fluid feed.

Advantageously, the assembly also includes a second device according to the invention mounted in parallel with the first device according to the invention.

According to another feature of the invention, the assembly includes a plurality of devices according to the invention mounted in parallel with one another.

Advantageously, the devices of the plurality can be identical or be dedicated to a different microorganism type.

According to another feature of the invention, the fluid outlet port can be connected to a recipient or otherwise.

According to a feature of the invention, the device or devices according to the invention can be seated in compartments in an industrial piping or other structure.

According to a feature of the invention, the device has a volumetric capacity enabling 100 mL of liquid to be analyzed.

### SHORT DESCRIPTION OF THE FIGURES

The invention can be better understood from the description below of an embodiment of the present invention, given as a non-limiting example and explained with reference to the attached schematic figures. The attached schematic figures are listed below:

FIG. 1 is a perspective view of the device for determining contamination according to the invention,

FIG. 2 is a median cross-section view of the device shown in FIG. 1,

FIG. 3 is a detailed view of FIG. 2,

FIG. 4 is a partial perspective view of the device according to the invention showing the support grille seated in the housing,

FIG. 5 is a perspective bottom view of the cover of the device according to the invention,

FIG. 6 is a perspective view of the inside of the housing of the device according to the invention,

FIG. 7 is a cross-section view of the cover and of the filtration member of the device according to the invention, and

FIG. 8 is a cross-section view of the housing of the device according to the invention.

FIG. 9 is a schematic view of a first step of a first usage mode of the device according to the invention,

FIG. 10 is a schematic view of a second step of the first usage mode of the device according to the invention,

FIG. 11 is a schematic view of a third step of the first usage mode of the device according to the invention,

FIG. 12 is a schematic view of a first step of a second usage mode of the device according to the invention,

FIG. 13 is a schematic view of a second step of the second usage mode of the device according to the invention,

FIG. 14 is a schematic view of a third step of the second usage mode of the device according to the invention.

### DETAILED DESCRIPTION OF THE FIGURES

The device 1 for determining contamination of a fluid by microorganisms according to the invention includes a housing 2, a cover 3, a filtration member 4, a nutrient layer 5, and a grille 6, as shown notably in FIG. 2.

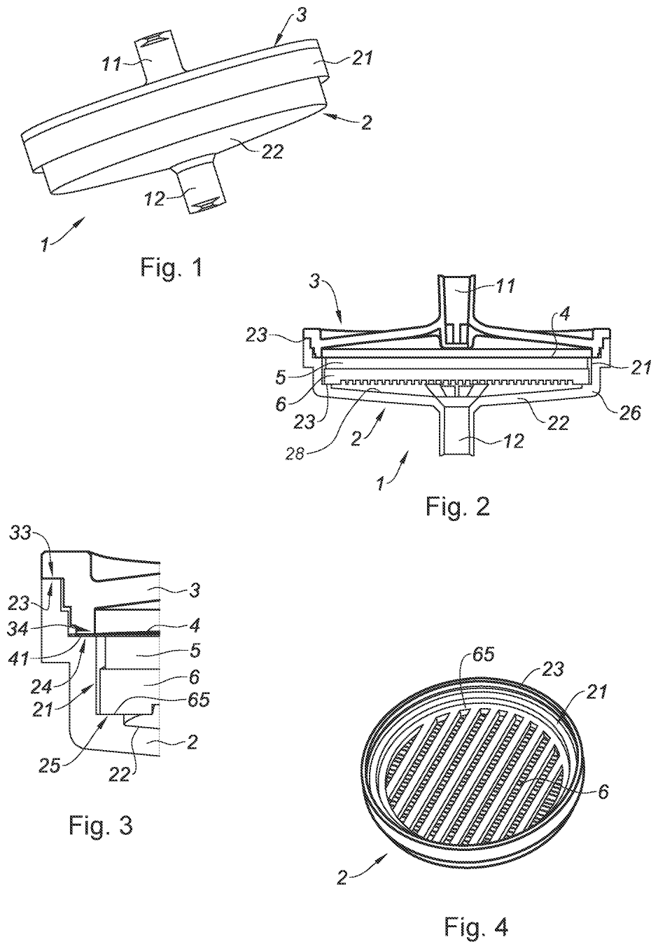

As shown in FIG. 1, only the cover 3 and the housing 2 are visible from the outside. Advantageously, the cover 3 and the housing 2 are sealed together by ultrasound. According to the cross-section shown in FIG. 2, the filtration member 4 is arranged in the internal volume of the housing 2, the nutrient layer 5 is arranged beneath the filtration member 3, preferably in contact with this latter and a support grille 6 is arranged between the nutrient layer 5 and the bottom 22 of the housing 2.

According to the invention, the filtration membrane 4 is permeable to fluids and in particular to a gas or to a liquid having a viscosity that enables microbiological filtration free of all solid particles.

The device 1 also includes a fluid inlet port 11 and a fluid outlet port 12, as shown in FIG. 1. The fluid inlet port 11 is positioned on the cover 3 and the fluid outlet port 12 is positioned on the housing 2 and preferably on the bottom of the housing 2. The fluid inlet port 11 and the fluid outlet port 12 open out into the internal volume of the housing 2, as shown in FIG. 2. When the inlet port and the fluid outlet port are blocked by the stoppers, the device according to the invention is fluid tight.

As shown in FIG. 2 for example, the fluid inlet port 11 projects into the internal volume of the housing 2 relative to the inner surface 31 of the cover 3. Preferably, the fluid inlet port 11 is arranged substantially in the center of the cover 3. The fluid inlet port has a first end 11a extending outside the cover 3 that is designed to cooperate with a stopper (not shown) and a second end 11b extending inside the internal volume, the second end being provided with at least one lateral orifice 11c opening out into the internal volume and oriented to at least partially spray the fluid towards the inner surface 31 of the cover 3.

As shown for example in FIG. 2, the fluid outlet port 12 has a first end 12a extending outside the housing 2 that is designed to cooperate with a stopper and a second end 12b that is flush with the bottom 22 of the housing 2.

The fluid inlet port 11 and the fluid outlet port 12 extend respectively along an axis substantially secant and preferably perpendicular to the cover 3 or to the bottom of the housing 2. In the example shown in FIG. 2, the fluid inlet port 11 and the fluid outlet port 12 are aligned and arranged opposite one another.

The housing 2 of the device 1 is described below in greater detail with reference to FIGS. 1, 2, 6 and 8. As shown in FIG. 1, the housing 2 is substantially cylindrical.

As shown in FIG. 2, the housing 2 has an internal volume delimited by at least one side wall 21 and a bottom 22.

FIGS. 2 and 8 show a cross section of the housing 2, which shows a first circumferential bearing surface 23 shaped to cooperate complementarily with an edge 33 of the cover 3. Furthermore, according to the invention, the housing 2 has at least one second bearing surface 24 extending in the internal volume and shaped to receive a peripheral portion 41 of the filtration member 4, said second bearing surface 24 being shaped to cooperate with a portion 34 of the cover 3 such that the peripheral portion 41 of the filtration member 4 is clamped between the cover 3 and the housing 2, as shown in detail in FIG. 3. Furthermore, the housing 2 has a third bearing surface 25 that is designed to receive the support grille 6 and more specifically to receive a peripheral edge 65 of the support grille 6. The third bearing surface 25 extends inside the internal volume of the housing 2 and is an external shoulder 26 of the housing 2, as shown in FIG. 8.

As shown in FIG. 8, each bearing surface 23, 24, 25 has a different diameter and advantageously the first bearing portion 23 has a diameter that is greater than the second bearing portion 24, which in turn has a diameter that is greater than the third bearing portion 25.

As clearly shown in FIG. 6, the bottom 22 of the housing 2 has a plurality of supporting ribs 27 designed to support the support grille 6 and to guide the fluid towards the fluid outlet port 12. The supporting ribs 27 are arranged radially relative to the fluid outlet port 12. The supporting ribs 27 project from the surface of the bottom 22 and extend substantially perpendicularly to said surface of the bottom 22, as shown in the cross section in FIG. 8.

As shown notably in FIG. 8, the bottom 22 of the housing 2 has a surface 28 extending radially about the fluid outlet port 12 up to the side wall 21 of the housing 2, said inner surface 28 being inclined and converging towards the fluid outlet port 12 to facilitate drainage of the fluid. In the example shown in FIGS. 2 and 8, the inclination β of the surface 28 of the bottom 22 is strictly less than 0° and is between −5° and −10°. The applicant has carried out tests demonstrating that if the bottom is flat, the fluid drains only partially or not at all.

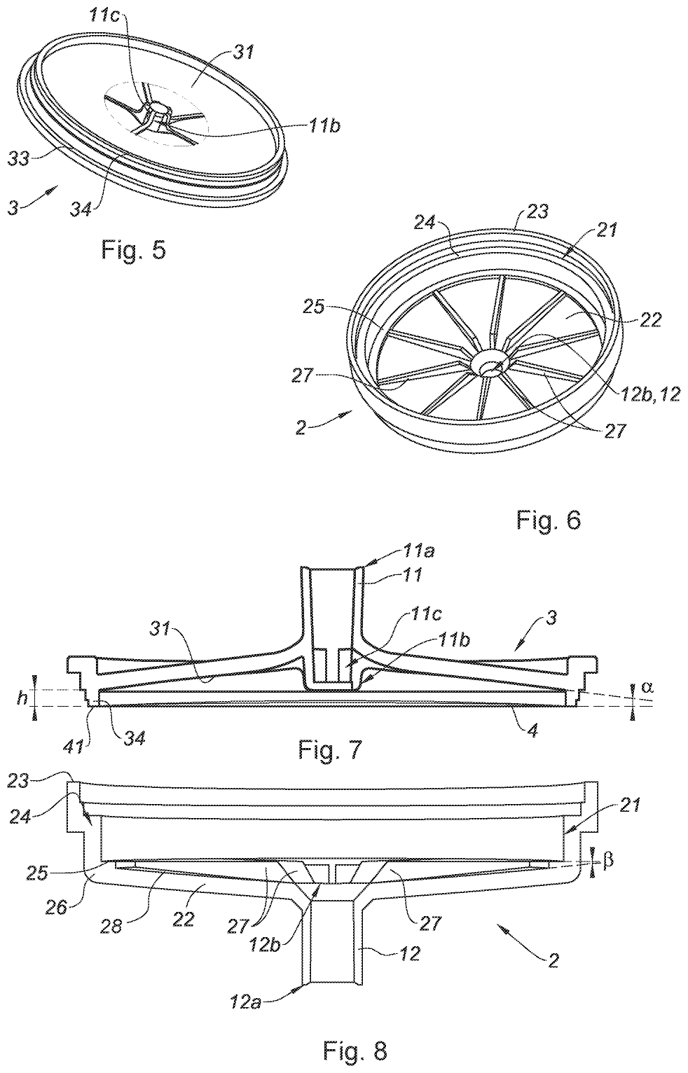

The cover 3 of the device 1 is described below in greater detail with reference to FIGS. 1, 2, 5 and 7.

As shown in FIG. 1, the cover 3 has a substantially cylindrical base surmounted by the fluid inlet port 11. The cover 3 has an inner surface 31 closing the internal volume of the housing 2. The inner surface of the cover 3 extends radially about the fluid inlet port 11 up to a peripheral edge 32 of the cover 3, said inner surface 31 being inclined and converging towards the fluid inlet port 11. The inner surface 31 therefore has an overall frustoconical shape extending from the fluid inlet port 11 and widening up to the peripheral edge 32 of the cover 3. In a variant that is not shown, the inner surface can be curved.

In the example shown in FIGS. 2 and 7, the inclination α of the inner surface 31 of the cover 3 is strictly greater than +4° and is between +5° and +15°. The applicant has carried out tests demonstrating that if the inner surface of the cover has an inclination equal to or less than 4°, the fluid is not uniformly distributed over the filtration member 4.

As shown in FIG. 7, the filtration member 4 is arranged in the housing 2 at a given distance h from the fluid inlet port 11. Advantageously, the given distance h is strictly greater than 1 mm, and preferably between 1.5 mm and 5 mm. Indeed, tests carried out by the applicant have demonstrated that the filtration member 4 has to be at a given distance h to enable the microorganisms to grow when the device 1 according to the invention is incubated. This given distance h is dependent on a minimum volume of air required to grow said microorganisms. In the example shown in FIGS. 1, 2, 5 and 7, the cover 3 is transparent or translucent, which enables growth of the microorganisms to be observed.

According to a feature of the invention, the support grille is designed to support the nutrient layer and the filtration member.

The support grille 6 of the device 1 is described below in greater detail with reference to FIGS. 2 and 4.

As shown in FIG. 2, the support grille 6 is arranged between the nutrient layer 5 and the bottom 22 of the housing 2. The support grille 6 has an overall cylindrical shape and is the same size as the nutrient layer 5.

As shown in FIG. 4, the support grille 6 has a plurality of through holes 61 distributed over the entire grille 6, which helps to distribute the fluid that has passed through the nutrient layer 5 over the entire surface 28 of the bottom 22 in order to drain said fluid as quickly as possible.

As shown in FIG. 2, the support grille 6 rests on the supporting ribs 27 of the housing 2. The support grille 6 has a peripheral edge 65 resting on the third bearing surface 25 of the housing 2, as shown in FIGS. 2 and 3.

In a variant that is not shown, the grille only rests on the third bearing surface 25 of the housing, and the supporting ribs are optional.

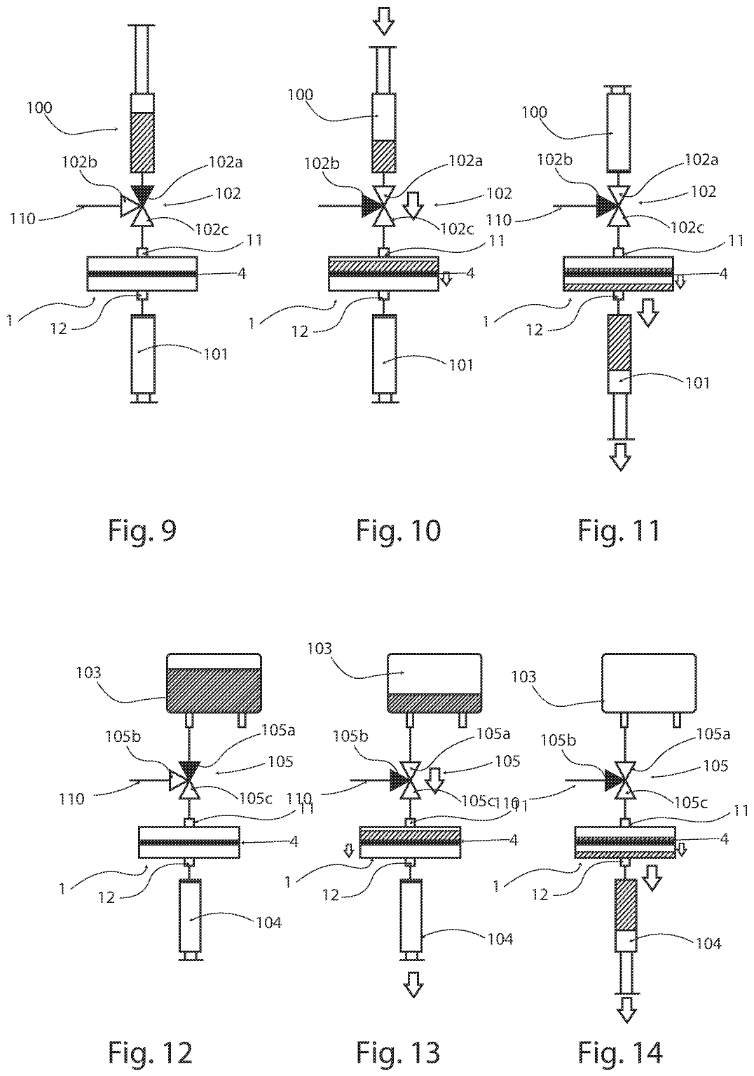

Two possible uses of the device according to the invention are described below with reference to FIGS. 9 to 14.

According to a first use illustrated in FIGS. 9 to 11, a syringe 100 or any other element including a piston containing the fluid to be analyzed is connected to the fluid inlet port 11 of the device 1, and a collector 101 or another syringe is connected to the fluid outlet port 12 of the device 1, as shown in FIG. 9. A three-way valve 102 is positioned between the device 1 and the syringe 100, the syringe 100 being connected indirectly to the fluid inlet port 11 of the device 1. Initially and as shown in FIG. 9, the first inlet 102a of the valve 102 is connected to the syringe 100 and closed, the second inlet 102b is open and connected to an air intake, and the outlet 102c is also open and connected to the fluid inlet port 11 of the device. This enables a vacuum to be created before the device is used and before the fluid to be analyzed/filtered is introduced.

Subsequently and as shown in FIG. 10, the first inlet 102a of the valve 102 is inserted and the fluid is injected into the device 1, the piston of the syringe 100 enabling the fluid to be pushed into the device 1, the second inlet 102b of the valve is then closed and the outlet 102c is open to enable the fluid to flow through the device 1.

Once all of the fluid to be analyzed has been injected through the device 1, the fluid is recovered in the collector 101, as shown in FIG. 11.

According to a second use illustrated in FIGS. 12 to 14, a tank 103 containing the fluid to be analyzed is connected to the fluid inlet port 11 of the device 1, and a syringe 104 or any other element including a piston and enabling aspiration of the fluid is connected to the fluid outlet port 12 of the device 1, as shown in FIG. 12. A three-way valve 105 is positioned between the device 1 and the syringe 100, the syringe 100 being connected indirectly to the fluid inlet port 11 of the device 1. Initially and as shown in FIG. 12, the first inlet 105a of the valve 102 is connected to the collector 103 and closed, the second inlet 105b is open and connected to an air intake, and the outlet 105c is also open and connected to the fluid inlet port 11 of the device 1.

Subsequently and as shown in FIG. 13, the first inlet 105a of the valve 105 is opened and the fluid contained in the collector 103 is aspirated by the syringe 104 positioned downstream of the device 1, the second inlet 105b of the valve is then closed and the outlet 105c is open to enable the fluid to flow through the device 1.

Once all of the fluid to be analyzed has been aspirated through the device 1, the fluid is recovered in the syringe 104, as shown in FIG. 14. Naturally, the capacity of the syringes and tanks or collectors is suitable for the volume of fluid to be analyzed.

Regardless of the use of the device described above, once the fluid has been injected (FIGS. 11 and 14), it is advantageous to inject sterile air via the air intake 110 to eliminate any stagnant water from the surface of the filtration member 4 of the device 1.

Indeed, the presence of fluid, even in small quantities, on the surface of the filtration member could result in the spread of bacterial colonies, false negatives or difficulties counting colonies after the incubation step. For this purpose, sterile air is aspirated by the syringe 100 or the tank 103 through the valve 102, 105, then injected into the device 1, in order to dry the surface of the filtration member 4.

The advantages of these uses are that these usage techniques are simple, they require no effort (notably the first use), the workflow lasts less than one minute in total, no laboratory infrastructure is required to carry out these operations, no staff qualified in microbiology are required, and the operation is very reproducible.

Naturally, the invention is not limited to the embodiments described and/or illustrated in the attached figures. Modifications may be made to the invention, notably in terms of the constitution of the different elements or by substitution for technical equivalents, without thereby moving outside the scope of protection of the invention.

1. A device for determining contamination of a fluid by a microorganism, said device comprising:
a housing having an internal volume delimited by at least one side wall and a bottom,
a cover closing the housing and positioned opposite the bottom,
a fluid inlet port arranged on the cover that opens out into the internal volume of said housing, the fluid inlet port extending along an axis that is substantially secant to the cover,
at least one filtration member arranged in the internal volume,
at least one nutrient layer comprising a microbiological culture medium,
wherein the device includes:
a fluid outlet port arranged on the housing that opens out into the internal volume of said housing, and in that the cover has an inner surface in the internal volume extending radially about the fluid inlet port up to the peripheral edge of the cover, said inner surface being inclined or curved and converging towards the fluid inlet port,
a support grille configured to support the nutrient layer
and in that the bottom of the housing has a surface extending radially about the fluid outlet port up to the side wall of the housing, said surface being inclined and converging towards the fluid outlet port.

2. The device as claimed in claim 1, in which the inclination of the inner surface of the cover is strictly greater than 4°.

3. The device as claimed in claim 1, in which the inclination of the surface of the bottom is strictly less than 0°.

4. The device as claimed in claim 1, in which the fluid inlet port projects into the internal volume of the housing relative to the inner surface of the cover.

5. The device as claimed in claim 1, in which the filtration member is arranged in the housing at a given distance h from the inlet port, the given distance h being strictly greater than 1 mm.

6. The device as claimed in claim 1, in which the housing has a first circumferential bearing surface shaped to cooperate complementarily with an edge of the cover.

7. The device as claimed in claim 1, in which the housing has at least one second bearing surface extending in the internal volume and shaped to receive a peripheral portion of the filtration member, said second bearing surface being shaped to cooperate with a portion of the cover such that the peripheral portion of the filtration member is clamped between the cover and the housing.

8. The device as claimed in claim 1, in which the bottom of the housing has supporting ribs arranged radially relative to the fluid outlet port, said ribs being designed to hold the support grille and to guide the fluid towards the fluid outlet port.

9. An assembly comprising at least one fluid feed and at least one device as claimed in claim 1, said device being connected by means of the fluid inlet port thereof to a fluid feed.
